(19) United States
(12) Patent Application Publication
(10) Pub. No.: US 2004/0013749 A1
(43) Pub. Date: Jan. 22, 2004

(54) Antioxidant and immune boosting composition and methods of using

(76) Inventors: D. Gary Young, Alpine, UT (US); Marc Schreuder, Provo, UT (US); Sue Chao, Salt Lake City, UT (US)

Correspondence Address: KRAMER & AMADO, P.C., 2001 JEFFERSON DAVIS HWY, SUITE 1101, ARLINGTON, VA, 22202, US

(21) Appl. No.: 10/199,092

(22) Filed: Jul. 22, 2002

(51) Int. Cl. A61K 35/78

(52) U.S. Cl. 424/732; 424/765; 424/769

(57) ABSTRACT

Compositions are shown having high ORAC scores correlating with antioxidant capabilities. Such a composition, when combined with other food products can either maintain or increase ORAC scores while enhancing flavor characteristics of the final food product. The compositions have also been shown to increase immune activity of a mammal.

### BACKGROUND OF THE INVENTION

1. Field of the Invention

This invention relates generally to an antioxidant composition comprising combinations of fruit and berry juices for reducing the harmful effects of free radical damage to mammals and methods of using said compositions for increasing immune function.

2. Description of Related Art

Disease and aging, according to an increasing number of research studies, is largely affected by the amount of free radicals present in the body. These free radicals are the result of normal metabolism and are electrically charged species that have the ability to disrupt many biological structures and processes. As these charged species travel through an organism, they try to balance their electrical charge and in turn disrupt or even destroy molecules in the body.

One of the most common types of free radicals are the reactive oxygen species (ROS). These are the products of normal cell respiration and metabolism and are generally regulated by antioxidants produced in the body. Due to environmental agents such as pollution, and lifestyle factors such as smoking or exercising, the production of free radicals is increased. Such increase may bring the body out of balance, especially as the body ages and the mechanisms that produce antioxidants lose their ability to produce these compounds at their necessary rate. This type of damage can range from disruption of biological processes, killing of cells, and most damaging of all, mutation of genetic material, which may lead to the occurrence of cancer.

In the market today there are many products that contain antioxidants at various levels. These come in the form of foods, liquids and nutritional supplements. The richest sources of these vital nutrients commonly are found in fruits and vegetables having compounds such as Vitamin C, Vitamin E, beta-Carotene and others. Antioxidants function to bind these free radicals and stabilize and scavenge them out of the system, thereby reducing the amount of damage they may cause.

Since many fruits and vegetables contain these vital nutrients, it became necessary to determine at what levels these nutrients were present in foods. Of greater importance is the ability of antioxidants in these foods to absorb free radicals. USDA Researchers at Tufts University developed a laboratory test know as ORAC (Oxygen Radical Absorbance Capacity) which rates different foods according to their antioxidant content and its ability to bind these free radicals. Through this test, different foods may be compared and analyzed for their antioxidant ability.

In correlation to the antioxidant abilities of nutrients, these may also have beneficial immune increasing activity. This may be shown through measurement of increased spleen weight and splenic phagocytes. The spleen being the major site of immune responses to blood-borne antigens may be effectively measured for increased weight and phagocyte cell counts in response to the addition of certain antioxidants to the diet of a mammal.

A negative drawback to the ingestion of these foods containing antioxidants is a common problem encountered by modern society's reliance on processed foods: their taste. This acquired aversion to consuming natural foods is derived from these foods having flavors that are not appetizing as compared to high sugar and/or high fat and oil content food products. Many of the food products found on the market today contain such high levels of sugars and/or fats that most human have become accustomed to their flavor. Once the habit of ingesting these modern foods is ingrained, it becomes difficult to consume healthier foods having more natural flavors. These natural flavors are derived from the chemical properties of the plants from which they are derived and have almost become foreign flavors to today's consumers. Due to the unappetizing flavor of many of the foods which are so vital for extended life and disease prevention, there is presently a need to combine food products in order to make these more appetizing.

Based on this knowledge, it is an object of the present invention to provide food products that have the ability to absorb free radicals at a desired rate and thereby prevent biological damage to a human.

It is another object of the present invention is to provide a method of reducing free radical damage through the use of compositions having high ORAC scores when combined.

It is yet another object of the present invention to provide a method of increasing immune activity, as measured by increased spleen weight and phagocyte cell counts.

It is a further object of the present invention to combine food products having high ORAC scores but unfavorable flavor characteristics with other food products having flavor characteristics that will combine and/or mask the unfavorable flavor characteristics as well as maintaining and/or increasing ORAC scores.

The foregoing objects and advantages of the invention are illustrative of those that can be achieved by the present invention and are not intended to be exhaustive or limiting of the possible advantages which can be realized. Thus, these and other objects and advantages of the invention will be apparent from the description herein or can be learned from practicing the invention, both as embodied herein or as modified in view of any variation which may be apparent to those skilled in the art. Accordingly, the present invention resides in the novel methods, arrangements, combinations and improvements herein shown and described.

### SUMMARY OF THE INVENTION

In light of the present need for antioxidants having a high degree of free radical binding capabilities, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

According to the present invention an antioxidant composition is shown, comprising at least one portion of a berry from the genus Lycium having an ORAC_{Total FL} of at least 25,000 and a food product, which in combination with the Lycium berry maintains or increases the ORAC_{Total FL} of the combination to 25,000 or above.

Additionally, a method is disclosed for increasing immune activity of a mammal by administering at least one portion of a berry from the genus Lycium having an ORAC_{Total FL} of at least 25,000 together with a food product which in combination with said berry maintains or increases the ORAC_{Total FL} of the combination to 25,000 or above.

Further, a method of improving the flavor characteristics of a food product containing at least one portion of a berry from the genus Lycium while maintaining high ORAC scores is disclosed. This is accomplished by providing at least one portion of a berry from the genus Lycium having an ORAC_{Total FL} of at least 25,000 together with a food product which in combination with said berry maintains or increases the ORAC_{Total FL} of the combination to 25,000 or above.

### DETAILED DESCRIPTION OF THE PREFERRED EMBODIMENTS OF THE INVENTION

In the present invention, a variety of compositions are shown that are useful for their high degree of free radical binding capabilities. These are measured according to the ORAC analysis performed by Brunswick Laboratories of Wareham, Mass. The ORAC analysis, which utilizes Flourescein as the fluorescent probe, provides a measure of the scavenging capacity of antioxidants against the peroxyl radical, which is one of the most common reactive oxygen species (ROS) found in the body. ORAC_{Hydro FL} reflects water-soluble antioxidant capacity and the \hat{ }ORAC_{Lipo FL} is the lipid soluble antioxidant capacity. ORAC_{Total FL} is the sum of ORAC_{Hydro FL} and ORAC_{Lipo FL} Trolox, water-soluble Vitamin E analog, is used as the calibration standard and the ORAC result is expressed as micromole Trolox equivalent (TE) per liter.

Using the ORAC analysis a variety of tests were performed and are summarized below:

TABLE I

| Sample | ORAC_{Hydro FL} | ORAC_{Lipo FL} | \hat{ }ORAC_{Total FL} |
|---|---|---|---|
| Wolfberry Juice 75% | 89,105 | 1,998 | 91,103 |
| Blueberry Juice 25% | | | |
| Wolfberry Juice 50% | 143,966 | 2,292 | 146,258 |
| Blueberry Juice 50% | | | |
| Wolfberry Juice 25% | 204,234 | 2,227 | 206,461 |
| Blueberry Juice 75% | | | |
| Wolfberry Juice 75% | 45,791 | 964 | 46,755 |
| Pomegranate Juice | | | |
| 25% | | | |
| Wolfberry Juice 50% | 60,198 | 1,087 | 61,285 |
| Pomegranate Juice | | | |
| 50% | | | |
| Wolfberry Juice 25% | 96,780 | 805 | 97,585 |
| Pomegranate Juice | | | |
| 75% | | | |
| Wolfberry Juice 75% | 91,257 | 2,251 | 93,508 |
| Raspberry Juice 25% | | | |
| Wolfberry Juice 50% | 170,668 | 2,799 | 173,467 |
| Raspberry Juice 50% | | | |
| Wolfberry Juice 25% | 293,759 | 2,862 | 296,621 |
| Raspberry Juice 75% | | | |
| Wolfberry Juice 75% | 35,724 | 1,452 | 37,194 |
| Apricot Juice 25% | | | |
| Wolfberry Juice 50% | 31,683 | 944 | 32,627 |
| Apricot Juice 50% | | | |
| Wolfberry Juice 25% | 37,263 | 457 | 37,720 |
| Apricot Juice 75% | | | |

As illustrated by TABLE II, Wolfberry (Lycium barbarum v. Ningxia) juice, alone shows a very high ORAC reading alone. This juice or portions of a berry from the genus Lycium would be very beneficial alone but suffer from the unfavorable flavor characteristics exhibited by Lycium alone. The benefits of Lycium can be taken advantage of through combining with other food products, especially fruits that enhance the flavor characteristics and make the ultimate product more enjoyable to consumers. For purposes of combining Lycium it is of the highest importance to combine with products that maintain its excellent antioxidant properties. For example, Wolfberry:apricot juice were combined at ratios of 25:75; 50:50; and 75:25. When compared to a total ORAC reading of 35,120 the addition of apricot juice had little effect and the ORAC readings for the three ratios only fluctuated between 32,627 and 37,720. Such a combination would produce a food product that is more appetizing to a consumer while allowing for the benefit of Lycium's high ORAC scores.

In contrast to the readings of the Wolfberry and apricot mixtures, Wolfberry in combination with blueberry had unexpected results based on what combinations of the readings shown on TABLE II would have produced. Wolfberry:blueberry combinations at 25:75; 50:50; and 75:25 showed increasing total ORAC readings of 91,103; 146,258 and 206,461. Based on these findings the antioxidant effectiveness and the ability of free radical binding of certain combinations of food products may be tailored to the particular needs of a person. Further, there is a large range of possibilities for flavor modification while achieving high ORAC scores.

TABLE II

| Sample | Total ORAC |
|---|---|
| Wolfberry | 35,120 |
| Pomegranates | 97,207 |
| Blueberries | 249,280 |
| Raspberries | 223,620 |

In correlation to the antioxidant activity of nutrients having high ORAC readings, immunity is also beneficially affected through their consumption. Historically, immunity has meant protection against both infectious and noninfectious diseases. The cells and molecules responsible for immunity constitute the immune system, and the collective and coordinated response to the introduction of foreign substances is called the immune response. Microbes and other foreign substances which illicit immune response can be categorized into two distinct types: the innate immune response and the adaptive immune response. The mechanism of innate immunity provides the initial defense against infections, and macrophages and granulocytes are a huge component of the innate immunity. They are phagocytes that are able to engulf and clear unrecognized foreign substances invading the body. They can also present these phagocytosed foreign antigens to lymphocytes, which are responsible for the actions of adaptive immunity. For the study described below with reference to TABLE III, splenic phagocytes were chosen for analysis since the spleen, as an organ is the major site of immune response to blood-borne antigens.

Wolfberryjuices and its combinations of various types of juices (Blueberry juice, Raspberry juice, Pomegranate juice) were injected intraperitoneally in to test groups of Swiss Weber mice, while the control groups received only equal volumes of sterilized saline (0.85% (w/v) NaCl). After eight days of receiving IP injections, the mice were sacrificed by inhalation euthanasia followed by cervical dislocation. All mice were weighed and their spleen harvested for the dynabead cell enrichment protocol described as follows.

Total cells from each spleen were collected and suspended in RPMI 1640 containing 10% fetal bovine serum (FBS). The erythrocytes present in the cell suspension were lysed by brief treatment with sterile aqueous 0.83% (w/v) ammonium chloride. For splenic phagocytes isolation, single cell suspensions were incubated with 0.5 μg/μl biotinylated anti-MAC-1 antibodies for 30 min. on ice. Following washing with 1640 RPMI medium, cells were resuspended with m-280 magnetic Dynabeads coated with streptavidin incubated at a bead:cell ratio of 1:1 for 10 min with agitation at 4 degrees Celsius. Cells bound to antibodies were depleted by two rounds of exposure to a magnetic field. Successfully depleted splenic phagocytes were collected and counted using a hematocytometer. The results can be seen in Table III.

TABLE III

| Composition of | | | Cell Counts |
|---|---|---|---|
| Intraperitoneal | Mice Body | Mice Spleen | (Splenic |
| Injection | Weight (G) | Weight (G) | Phagocytes × 10⁶) |
| Saline | 26.48 | 0.0973 | 13.63 |
| Wolfberry Juice | 24.93 | 0.1489 | 22.32 |
| Wolfberry Juice + | 22.90 | 0.1427 | 28.70 |
| Blueberry | | | |
| 50:50 (w:w) | | | |
| Wolfberry Juice + | 24.60 | 0.1694 | 36.20 |
| Raspberry | | | |
| 50:50 (w:w) | | | |
| Wolfberry Juice + | 24.30 | 0.1409 | 19.88 |
| Pomegranate | | | |

As illustrated by TABLE III, the results of the study give clear indication of the ability of compounds having high ORAC scores in increasing the weight of the spleen, and correlating with the observed expansion of the population of splenic phagocytes in comparison to the control group. As previously described, phagocytes are a crucial component to the body's innate defenses by phagocytosis of foreign substances that invade the body, possession of a large population of phagocytes will enable the body to increase the speed of elimination of foreign substances within the body and thus prevent the development of potential illness. Another correlation discovered by the inventor's is the positive correlation found between increased immune function and ORAC scores. Through the ORAC method of analysis, these scores not only yield the antioxidant ability of a food product, but also provide a measurement of increased immune activity according to increased spleen weight and splenic phagocyte cell counts. According to this method, foods may be measured for ORAC scores, combined to achieve the desired number and then administered to immuno-compromised subjects according to their needs. Such method could yield important benefits to the medical community in dealing with patients having diseases affecting immunity and immune function. For example, a food product may be measured using ORAC analysis to yield and ORAC score. The ORAC score may then be correlated with the results of TABLE III to derive a predicted increase in spleen weight and splenic cell count. The food product may then be tailored to the particular needs of the person, based on their need for immunity modulation.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

What is claimed is:

1. An antioxidant composition, comprising:
at least one portion of a berry from the genus Lycium having an ORAC_{Total FL} of at least 25,000; and
a food product which in combination with said berry maintains or increases the ORAC_{Total FL} of the combination to 25,000 or above.

2. An antioxidant composition according to claim 1, wherein the food product is blueberry and extracts thereof.

3. An antioxidant composition according to claim 1, wherein the food product is raspberry and extracts thereof.

4. An antioxidant composition according to claim 1, wherein the food product is pomegranate and extracts thereof.

5X. A method of increasing immune activity of a mammal, comprising:
administering at least one portion of a berry from the genus Lycium having an ORAC_{Total FL} of at least 25,000 together with a food product which in combination with said berry maintains or increases the ORAC_{Total FL} of the combination to 25,000 or above.

X. A method according to claim X, wherein the food product is blueberry and extracts thereof.

X-1. A method according to claim X, wherein the food product is raspberry and extracts thereof.

X-2. A method according to claim X; wherein the food product is pomegranate and extracts thereof.

XX. A method of improving the flavor characteristics of a food product containing at least one portion of a berry from the genus Lycium while maintaining high ORAC scores, comprising:
providing at least one portion of a berry from the genus Lycium having an ORAC_{Total FL} of at least 25,000 together with a food product which in combination with said berry maintains or increases the ORAC_{Total FL} of the combination to 25,000 or above.

XX-1. A method according to claim XX, wherein the food product is blueberry and extracts thereof.

XX-2. A method according to claim XX, wherein the food product is raspberry and extracts thereof.

XX-3. A method according to claim XX, wherein the food product is pomegranate and extracts thereof.

XXX. A method of predicting increased immune activity, comprising:
testing a food product using the ORAC analysis to yield and ORAC score; and
correlating said ORAC score to a predicted increase in spleen weight and splenic cell count.

XXX-1. A method according to claim XXX, further comprising administering said food product to a patient in need thereof.
